(19) United States
(12) Patent Application Publication
(10) Pub. No.: US 2021/0038768 A1
(43) Pub. Date: Feb. 11, 2021

(54) SHEET FOR COVERING WOUND, AND METHOD FOR COVERING WOUND

(71) Applicants: TOKYO WOMEN'S MEDICAL UNIVERSITY, Tokyo (JP); CellSeed Inc., Tokyo (JP)

(72) Inventors: Takeshi OHKI, Tokyo (JP); Masayuki YAMATO, Tokyo (JP); Tatsuya SHIMIZU, Tokyo (JP); Masakazu YAMAMOTO, Tokyo (JP)

(73) Assignees: TOKYO WOMEN'S MEDICAL UNIVERSITY, Tokyo (JP); CellSeed Inc., Tokyo (JP)

(21) Appl. No.: 16/983,232

(22) Filed: Aug. 3, 2020

(30) Foreign Application Priority Data
Aug. 8, 2019 (JP) 2019-146319

(51) Int. Cl. A61L 27/38; A61L 27/36

(52) U.S. Cl. CPC A61L 27/3891; A61L 27/3604; A61L 27/3834

(57) ABSTRACT

A sheet for covering a wound includes a laminate of a serosal membrane and a cell sheet. The sheet for covering a wound has a proper thickness and strength. The sheet does not flow out from the wound site and can be fixed to the wound site by suture or anastomosis, if necessary. The sheet can be stably engrafted onto a wound region.

### TECHNICAL FIELD

The present technology relates to a sheet for covering a wound. In particular, the present technology relates to a sheet that is to be engrafted into a wound region to cover the wound and a method of covering a wound using the sheet.

### BACKGROUND ART

After surrounding tissues including lesions are excised to treat a variety of visceral diseases, such as malignancies, the remaining portions of the organs must be sutured or inosculated. A severe problem associated with surgery is failure of the sutures. For example, malignancies infiltrate deep tissues or surrounding tissues to spread lesions over wide areas in progressive cancer of digestive tracts; hence, the surrounding tissues including lesions must be excised, and then the remaining portions of digestive tracts must be sutured or inosculated. Failure of the sutures after gastrointestinal tract surgery will cause severe problems.

The failure of the sutures after the gastrointestinal tract surgery indicates partial or entire dehiscence of the anastomotic site of the digestive tract due to impaired healing and is one of the complications that involve exsorption of intestinal tract contents toward the pleural space and the abdominal cavity. This exsorption will sometimes cause potentially fatal infectious disorders. The failure of the sutures will lead to various affects, such as a decrease in quality of life (QOL), e.g. fever onset, abdominal pain, and impaired nutrition and muscle weakness due to an extended period to start of ordinary meals; development of bowel blockage or other complications; medical economic problems due to extended hospitalization periods; and prognosis, e.g. postoperative mortality due to septic shock, and risk of relapse.

The failure of the sutures is caused by impediment to healing or repair of tissues at anastomotic sites. Factors that impede healing during restoration periods include systemic factors, such as low nutrient preoperative conditions, administration of medicines, e.g. steroid, and chronic disorders, e.g. diabetic mellitus, hepatic disorder, and renal disorder; and regional factors, such as hematogenous disorder at anastomotic sites and their peripheries, hypertonicity, and infectious diseases.

In order to prevent the failure of the sutures, many surgeons have make their best efforts to operations of patients after their preoperative systemic conditions are improved, development of automatic anastomosis devices, selection of appropriate pretreatments and anastomosis depending on surgical sites and states, a reduction in stress at anastomotic sites, establishment of proper bloodstreams, insertion of drains, and improvements in techniques, such as decompression, and surgical instruments. Nevertheless, the rate of the failure of the sutures is as relatively high as 3 to 14%. In particular, the risk of failure of the sutures increases as the cancer lies at a lower site. Increasing cases of current preoperative chemoradiotherapy lead to increases in risks of failure of the sutures. In order to prevent gastrointestinal suture failure, there are not a few cases where temporary colostomy is unavoidable in exchange for decreased QOL.

Animal experiments have been reported using mesenchymal stem cells and adipose-derived stem cells that can promotes tissue wound healing to solve such a problem, and the major portion of the transplant procedures have been cell infusions, such as intravenous injection and local injection. In general, the process of wound healing at anastomotic sites of digestive tract includes an inflammation term up to postoperative days 3 to 4 and a repair term up to postoperative days 7. Collagenase derived from inflammation cells decomposes existing collagen to reconstruct submucosal tissues at the anastomotic sites during the inflammation term on postoperative days 3 to 4, and fibroblasts producing collagen proliferate to increase the collagen and to keep the continuity and physical high tension of the tissues during the repair term up to postoperative days 7. The implantation process involving injection of cells into the anastomotic sites barely maintains cells in situ and the infused cells are exposed to collagenase, which is a proteinase, during the inflammation term; hence, cellular disorder by collagenase tends to decrease the tissue repair.

WO2017/130802 discloses a technique involving applying a cell sheet composition including mesenchymal stem cells onto a wound region of a hollow organ to prevent the exsorption of contents from the wound region of the hollow organ.

### SUMMARY OF INVENTION

### Technical Problem

A technique has been developed that involves applying a cell sheet composition including mesenchymal stem cells onto a wound region of a hollow organ to prevent the exsorption of contents from the wound region of the hollow organ, as described above. Since the cell sheet is thin and fragile, it is disadvantageous in difficulty in handling during implantation in clinical practice at present. In particular, laparoscopic techniques have recently become popular in colon elective surgeries worldwide. It is significantly difficult to implant a fragile cell sheet by forceps operations.

After a lapse of several days from implantation of the cell sheet into the wound region, the cell sheet may sometimes flow out from the wound site to impair the preventive effect of exsorption of contents from the wound region of the hollow organ.

The main object of the present technology is to provide a sheet for covering a wound that can be stably engrafted onto the wound region with enhanced manipulation performance.

### Solving Means

The inventors, who have conducted extensive studies on the structure of a sheet for covering a wound to solve the problems of the conventional art, have produced a sheet for covering a wound that can be stably engrafted onto the wound region with enhanced manipulation performance in combination with a certain biological membrane to complete the present technology.

The present technology provides a sheet to be engrafted into a wound region to cover the wound, the sheet comprising:

a serosal membrane having an implant face; and

a cell sheet laminated onto the implant face of the serosal membrane.

The serosal membrane of the sheet for covering a wound may be a peritoneal membrane.

The sheet for covering a wound may be applied to a sutured or inosculated wound region.

The sheet for covering a wound may be applied to a wound region of a hollow organ.

The sheet for covering a wound may be engrafted into the outer wall of a hollow organ.

The present technology also provides a method of covering a wound comprising engrafting a sheet for wound therapy into a wound region, wherein the sheet includes a laminate of a serosal membrane and a cell sheet.

In the method of covering a wound of the present technology, the sheet for wound therapy may be engrafted by suturation to the wound region.

The serosal membrane used in the method of covering a wound of the present invention may comprise a peritoneal membrane.

In the method of covering a wound of the present technology, the sutured or inosculated wound region can be covered.

In the method of covering a wound of the present technology, a wound region of a hollow organ can be covered.

In the method, the sheet for covering a wound can be engrafted into the outer wall of a hollow organ.

### Advantageous Effects

The sheet for covering a wound of the present technology is a laminate of a cell sheet and a serosal membrane and has a proper thickness and strength; hence, the sheet can be readily engrafted into a target site with a stable operation. The sheet does not flow out from the wound site and can be fixed to the wound site by suture or anastomosis, if necessary; hence, the sheet can be stably engrafted onto a wound region.

### BRIEF DESCRIPTION OF DRAWINGS

FIG. 1 includes photographs of back muscles immediately after a sheet for covering wound, only a cell sheet, and only a peritoneal membrane are engrafted.

FIG. 2 includes photographs of back muscles three days after the sheet for covering wound, only the cell sheet, and only the peritoneal membrane are engrafted.

FIG. 3 includes photographs of back muscles seven days after the sheet for covering wound, only the cell sheet, and only the peritoneal membrane are engrafted.

FIG. 4 includes microscopic photographs of tissue sections that are stained with hematoxylin-eosin three days and seven days after the operation.

FIG. 5 includes microscopic photographs of tissue sections that are stained with a green fluorescent protein three days and seven days after the operation.

FIG. 6 includes microscopic photographs of tissue sections that are stained with calretinin three days and seven days after the operation.

### EMBODIMENT OF INVENTION

Preferred embodiments for performing the present technology will now be described. The following embodiments are typical examples of the present technology and should not be construed to limit the scope of the present technology.

The sheet of the present technology is to be engrafted into a wound region to cover the wound and includes a serosal membrane (1) having an implant face and a cell sheet (2) laminated onto the implant face of the serosal membrane. The sheet for covering a wound of the present technology, which is a laminate of the cell sheet and the serosal membrane, has a proper thickness and strength; hence, it can be readily engrafted into a target site with a stable operation. The sheet does not flow out from the wound site and can be fixed to the wound site by suture or anastomosis, if necessary; hence, it can be successfully engrafted onto a wound region in a stable state.

When the sheet for covering a wound of the present technology is engrafted into a sutured or inosculated wound region, failure of the sutures can be more effectively prevented in comparison with implantation of only a cell sheet is engrafted into the wound region. When the sheet for covering a wound of the present technology is engrafted into a wound region of a hollow organ, the exsorption of contents from the wound region of the hollow organ can be more effectively prevent in comparison with implantation of only a cell sheet into the wound region.

The lamination of the serosal membrane (1) and the cell sheet (2) of the present technology may be performed by any process. Examples of the process include direct lamination of the serosal membrane (1) and the cell sheet (2); adhesive lamination of the serosal membrane (1), an adhesive layer, and the cell sheet (2); and cultivation of target cells on the serosal membrane (1) to form a cell sheet on the serosal membrane (1).

In the present technology, the sheet for covering a wound can be prepared by lamination of a preliminarily harvested serosal membrane and a cell sheet. Alternatively, an autologous serosal membrane collected from a subject during a surgery may be used. For example, a serosal membrane such as a peritoneal membrane collected during a surgery and a preliminarily prepared cell sheet may be laminated to form a sheet for covering a wound region of a hollow organ in situ for implantation of the sheet onto a wound region during the surgery.

The sheet for covering a wound of the present technology can be engrafted into any wound region, preferably a sutured or inosculated wound region. In the sutured or inosculated site, the inflamed tissues in the wound are in close contact with each other; hence, the sheet for covering a wound of the present technology engrafted into the wound region can facilitate the healing of the wound region. The sheet for covering a wound of the present technology can enhance the production of collagen to accelerate reconstruction and thus healing in the wound site and its periphery.

The term “wound” in the present technology refers to a wound state of a tissue or organ in a broad sense. Examples of the wound include, thermal injuries, pressure ulcer sores, contused wounds, incision wounds, abraded wounds, ulcers, surgical wounds, gunshot wounds, explosive injuries, stab wounds, impalement injuries, and bite wounds. The sheet of the present technology is suitable for incision wounds of organs, surgical wounds, stab wounds, impalement injuries, and bite wounds, most suitable for surgical wounds. The term “surgical wound” in the present technology refers to a wound formed by the use of surgical knives, surgical scissors, medical lasers, forceps, and snares during a surgical operation. Any wound formed by the use of other surgical instruments can also be treated. The term “wound region” in the present technology includes the wound site itself and its surrounding tissues of the operated tissue or organs. The surrounding tissues lye, for example, within a 10 cm, 8 cm, 5 cm, 3 cm, or 2 cm radius from the center of the wound site. The wound region has any area and shape depending on a moment-to-moment basis.

The sheet for covering a wound of the present technology can be favorably applied to wound regions of hollow organs. After the sheet for covering a wound of the present technology is applied to a wound region, the wound region can withstand an increase in internal pressure of the hollow organ as a result of the healing effect, and thus can prevent the exsorption of contents from the wound region of the hollow organ.

The sheet for covering a wound of the present technology can be engrafted into the inner and/or outer wall of a hollow organ. The outer wall is preferred because the sheet can be readily engrafted therein in a surgical operation.

The term “hollow organ” in the present technology refers to an organ having a luminal structure. Examples of the hollow organ include digestive tracts, blood vessels, a bladder, and a vaginal. Preferred are digestive tracts. The term “digestive tract” in the present technology refers to organs from the buccal to the anus, for example, esophagus, stomach, small intestine, and colon.

The term “exsorption” from a wound region of a hollow organ in the present technology refers to leakage of the contents in the hollow organ to the exterior of the hollow organ. In the case that the hollow organ is digestive tract, the term refers to leakage of solid contents, such as food and its digested material; fluid contents, such as gastric fluid and buccal secretion; and gas contents, such as air, from the wound region into the pleural space and the abdominal cavity.

If the cancer occurring in a hollow organ, for example, a digestive tract infiltrates a deep layer, part of the digestive tract must be excised. In such a case, the remaining digestive tract must be sutured or inosculated. As described above, the failure of the sutures involving the exsorption of contents may occur at the sutured or inosculated site of the digestive tract for a variety of reasons. Since the sheet for covering a wound of the present technology is engrafted into the sutured or inosculated wound region of a hollow organ, in particular, a digestive tract, the wound region can be readily healed without failure of the sutures.

The sheet for covering a wound of the present technology exhibits a significantly high strength to pressure in comparison with implantation of a cell sheet alone into a hollow organ, and thus high ability to prevent the failure of the sutures of a digestive tract occurring by a variety of factors during a healing process (repair term) of the anastomosed tissue. The sheet of the present technology can accordingly contribute to a reduction in social loss, such as low QOL of patients, caused by additional therapies, e.g., colostomy and enterostomy, and consumption of various medical resources caused by failure of the sutures.

The sheet for covering a wound of the present technology does not flow out from the wound site as described above, and can be fixed to the wound site by suture or anastomosis, if necessary; hence it can be successfully engrafted to the wound region in a stable state. The sheet for covering a wound of the present technology can be sutured or inosculated onto wound regions by any process conventionally used in surgical operations. The sheet for covering a wound of the present technology may be sutured or inosculated onto the wound region with any soluble or insoluble suture thread. Insoluble suture threads are preferred in view of invasiveness. The thickness of the thread can be appropriately determined depending on the dimensions and site of the wound.

The serosal membrane (1) and cell sheet (2) will now be described in detail.

### (1) Serosal Membrane

The serosal membrane used in the sheet for covering a wound of the present technology can be collected from living organisms, for example, peritoneal membranes, pleurals, pericardials that are appropriately selected depending on the position of the wound region within the scope of the present technology.

The serosal membrane may be derived from mammal animals, such as human, rat, mouse, guinea pig, marmoset, rabbit, canine, cat, sheep, pig, goat, monkey, chimpanzee, and immunodeficient animals thereof; birds, reptile, amphibia, fishes, and insects. In the sheet for covering a wound of the present technology, human-derived serosal membranes are preferred in use for human therapy, pig-derived serosal membranes for pig therapy, monkey-derived serosal membranes for monkey therapy, and chimpanzee-derived serosal membranes for chimpanzee therapy. In the case of human therapy, the serosal membranes may be collected from the patients themselves (autogenous transplantation) or other persons (allograft).

### (2) Cell Sheet

The sheet for covering a wound of the present technology may be any common cell sheet that can be appropriately selected depending on the position of the wound region within the scope of the present technology. In specific, the sheet may be a monolayer or multilayer cell sheet that can be prepared through cultivation on a cell-cultivating substrate and then detachment from the substrate.

The cell sheet can be prepared as follows: Cells are cultivated on a stimuli-responsive cultureware covered with a polymer the surface of which has undergone modification of the molecular structure by thermal, pH, or optical stimulation, and the resulting cell sheet is separated from the modified stimuli-responsive cultureware while the adhesive state is kept among cells. Alternatively, cells are cultivated on a proper cultureware and the cell sheet is physically removed from an edge of the cultureware with a pair of tweezers. In a preferred embodiment, the stimuli-responsive cultureware is a temperature-responsive cultureware covered with a polymer having variable hydration force within a temperature range of 0 to 80° C. In detail, cells are cultivated on the temperature-responsive cultureware covered with a polymer in a broth at a temperature causing low hydration force in the polymer and then at a different temperature causing high hydration force, and are recovered from the cultureware into a sheet. The temperature causing low hydration force usually ranges from 33° C. to 40° C. The polymer applied onto the temperature-responsive cultureware may be either homopolymer or copolymer.

Atypical temperature-responsive polymer used in temperature-responsive culture dishes is poly(N-isopropylacrylamide), which has a lower critical melting temperature at 31° C. This polymer in a free form is dehydrated at a temperature above 31° C. in water into a turbid state due to coagulation of the polymer chains. In contrast, the polymer chains are hydrated at a temperature below 31° C. and present in the form of an aqueous solution. In the present technology, the polymer is fixed onto the surface of a cultureware such as a petri dish. At a temperature above 3120 C., the polymer chains on the cultureware are also dehydrated and are fixed onto the surface of the cultureware; hence, the surface of the cultureware is hydrophobic. At a temperature below 31° C., the polymer chains on the surface of the cultureware are hydrated and the surface of the cultureware covered with a polymer chain changes into a hydrophilic state. In such a state, the hydrophobic surface allows adhesion and proliferation of cells whereas the hydrophilic surface does not allow adhesion of cells. When the cultureware is cooled to a temperature below 31° C., the cells can be separated from the surface of the cultureware. After the cells are confluently incubated over the entire surface of the cultureware, the cultureware is cooled to a temperature below 31° C. to recover the cell sheet. Any temperature-responsive culture dish is available. Atypical example of the dish is UpCell (registered trademark) available from CellSeed Inc.

Cells used in the cell sheet of the present technology are collected from any animal. Examples of the animal include mammal animals, such as human, rat, mouse, guinea pig, marmoset, rabbit, canine, cat, sheep, pig, goat, monkey, chimpanzee, and immunodeficient animals thereof; birds, reptile, amphibia, fishes, and insects. In the sheet for covering a wound of the present technology, human-derived serosal membranes are preferred in use for human therapy, pig-derived cells for pig therapy, monkey-derived cells for monkey therapy, and chimpanzee-derived cells for chimpanzee therapy. In the case of human therapy, the cells may be collected from the patients themselves (autogenous transplantation) or other persons (allograft), or may be commercially-available cell lines.

Examples of the cell used in the cell sheet of the present technology include germ cells, such as sperm cells and ovum cytoplasm; somatic cells, stem cells, and precursor cells of organisms; extracorporeally stable cells (cell lines) having immortalization potential isolated from organisms; cells isolated from organisms and then artificially modified, and cells isolated from organisms and then undergone artificial nuclear exchange.

It is preferred to use mesenchymal stem cells in the present technology. The mesenchymal stem cells can be isolated by a known process from tissues of bone marrow, fat tissues, umbilical cord blood, tooth pulp, synovial, and placenta in organisms.

For example, hematopoietic cells are isolated by density gradient centrifugation from bone marrow fluid collected from bone marrow, are seeded onto a plastic incubation dish, and cultured at 37° C. in a 5% CO₂ environment to prepare mesenchymal stem cells derived from bone marrow.

Mesenchymal stem cells derived from fat tissues (adipose-derived stem cells) are prepared as follows: Collected fat tissues are minced, and digested with a collagenase type II at 37° C. for one hour. A culture medium is added to the tissues and subjected to centrifugal separation. The cell precipitation is rinsed with a basic culture medium, and is separated by filtration through a mesh such as a cell strainer. The cells are seeded onto a plastic incubation dish and are cultivated at 37° C. in a 5% CO₂ environment into adherent cells, which are then isolated. Mesenchymal stem cells derived from other tissues can be isolated by any known process without limitation.

The mesenchymal stem cells may be prepared by differentiation of pluripotent stem cells. The pluripotent stem cells in the present technology have replication competence and pluripotency and can form every cell constituting the body. The replication competence represents ability to produce two indifferent cells from one cell. Examples of the pluripotent stem cell usable in the present technology include embryonic stem cells, embryonic carcinoma cells (EC cells), trophoblast stem cells (TS cells), epiblast stem cells (EpiS cells), embryonic germ cells (EG cells), multipotent germline stem cells (mGS cells), and induced pluripotent stem cells (iPS cells).

Any mesenchymal stem cell can be used in the present technology. Preferred mesenchymal stem cells are ones derived from bone marrow or fat tissues because the process of collecting and isolating the mesenchymal stem cells from bone marrow or fat tissues has been well established. Fat tissues are preferred to bone marrow because a larger number of mesenchymal stem cells can be collected from fat tissues than from bone marrow.

Besides the mesenchymal stem cells, the cell sheet of the present technology may contain other cells, for example, vascular endothelial cells, vascular endothelial precursor cells, fibroblast cells, epithelial cells, and stromal cells, which can be appropriately selected depending on the site and purpose of implantation and used in combination with mesenchymal stem cells. The cell sheet may also contain cells derived from tissues from which the mesenchymal stem cells are collected.

The number of cells contained in the cell sheet in the present technology can depends on the size and severity of the wound region. The cell sheet may contain any level of cells, for example, at least 30%, at least 40%, at least 50%, at least 55%, at least 60%, at least 65%, at least 70%, at least 75%, at least 80%, at least 85%, at least 90%, at least 93%, at least 95%, at least 97%, at least 98%, at least 99% cells. As the cell content in the cell sheet increases, the coverage of the wound region also increases. When the sheet for covering a wound of the present technology is applied to a sutured or inosculated wound region, the failure of the sutures can be more effectively prevented. When the sheet for covering a wound of the present technology is applied to a wound region of a hollow organ, the exsorption of contents from the wound region of the hollow organ can be more effectively prevented.

In the present technology, the number of seeded cells for preparation of a cell sheet depends on the types of the animal and cell, and may be, for example, 0.3×10⁴ to 10×10⁶ cells/cm², 0.5×10⁴ to 8×10⁶ cells/cm², or 0.7×10⁴ to 5×10⁶ cells/cm². In the present technology, the temperature-responsive cultureware in which cells are cultured to confluent or subconfluent is controlled to a temperature that is higher than the upper critical melting temperature or lower than the lower critical melting temperature of the coating polymer and then the cell sheet is separated from the cultureware. In this process, the cell sheet can be prepared in a broth or any other isotonic solution, which can be selected depending on the purpose. Measures of facilitating the separation of the cell sheet includes tapping and wobbling of the cultureware, agitation of the culture medium with a pipette, and use of a pair of tweezers, which may be used alone or in combination. Conditions other than the temperature may follow routine procedures. For example, the culture medium to be used may contain a known serum, such as a fetal bovine serum (FBS), or may be free of serum.

In the production of the cell sheet of the present technology, the cell cultureware may have any form, for example, a dish, multiplate, flask, or flat sheet membrane. Examples of material for the cell cultureware include glasses, modified glasses, polystyrene, poly(methyl methacrylate), polycarbonate, and any other polymers and ceramics, which are usually used in cell incubation.

In the production of the cell sheet in the present technology, the cell cultureware may have two types of regions, i.e., cell adherent regions and cell nonadherent regions on its cultureware. For example, a cell cultureware having circular cell adherent regions and cell nonadherent regions on its culture surface enables two or more cell sheets to be prepared at the same time. In such a case, cell adherent region may have any shape and dimensions depending on the purpose. Example of the shape include circle, square, triangle, and rectangle. The cell nonadherent region may be provided by any means, for example, application of a polymer with low affinity to cells, such as a hydrophilic polymer, e.g., poly-N-acryloylmorpholine, polyacrylamide, poly(dimethyl acrylamide), poly(ethylene glycol), or cellulose, or a highly hydrophobic polymer, e.g., a silicone polymer and fluorine polymer.

The cell sheet can be separated from the cell cultureware by any means that meets the purpose of the present technology. For example, use of an enzyme is common for separation and recovery. Cultivation of cells on a stimuli-responsive cultureware is preferred because the sheet can be separated without damage.

The cell sheet used in the sheet for covering a wound of the present technology may be a laminated cell sheet including two or more cell sheets. The laminated cell sheet can carry an increased number of cells and thus can enhance the coverage of the wound region. For example, the sheet for covering a wound of the present technology used in a sutured or inosculated wound region can effectively prevent the failure of the sutures. The sheet for covering a wound of the present technology used in a wound region of a hollow organ can effectively prevent the exsorption of contents from a wound region of a hollow organ, resulting in an improvement in therapeutic effect.

The laminated cell sheet can be prepared, for example, as follows: A cell sheet floating in a broth is taken with a pipette, is placed onto another cell sheet on an incubation dish, and is laminated on the cell sheet by fluidity of the broth or with a cell spreading tool. Preferred is use of a cell spreading tool that can laminate the cell sheets without damage of the cell sheets. Any cell spreading tool that can trap the cell sheet may be used. Examples of the material for such a tool include poly(vinylidene difluoride) (PVDF), silicone resin, poly(vinyl alcohol), polyurethane, cellulose and its derivatives, chitin, chitosan, collagen, gelatin, and fibrin gels. The cell spreading tool may have any shape. Examples of the shape include stamp, membrane, porous membranous, non-woven fabric, and woven fabric. In some embodiments of the present technology, the cell spreading tool can recover the cell sheet without damage and overlay the cell sheet on another cell sheet. The cell spreading tool preferably includes a cell adherent portion composed of, for example, a cell adherent protein, a cell adherent peptide, or one or more hydrophilic polymers. Preferred is a stamp-type cell spreading tool having a cell adherent portion. The stamp-type cell spreading tool can prevent damage of the cell sheet and allows the cell sheet to be recovered without shrinkage from the incubation dish. The cell spreading tool can readily transfer a cell sheet onto another cell sheet, and thus laminate these cell sheets without shrinkage. The resulting laminated cell sheet thereby has a highly densified three-dimensional structure without gaps.

In the preparation of the cell sheet of the present technology, cells may be cultivated in a culture medium containing ascorbic acid (refer to Kato Y, et al. Allogeneic Transplantation of an Adipose-Derived Stem Cell Sheet Combined With Artificial Skin Accelerates Wound Healing in a Rat Wound Model of Type 2 Diabetes and Obesity. Diabetes. 2015 August; 64 (8): 2723-34). The cell sheet prepared in a culture medium containing ascorbic acid has higher tear strength than that in a culture medium free of ascorbic acid. The resulting cell sheet is accordingly suitable for implantation.

The cell sheet for covering a wound of the present technology may further contain any angiogenic factor. Examples of the angiogenic factor include a vascular endothelial growth factor (VEGF), a fibroblast growth factor (FGF), angiopoietin, a platelet derived growth factor (PDGF), transforming growth factor-β (TGF-β), matrix metalloprotease (MMP), VE-cadherin, ephrin, plasminogen activator, inducible nitrogen monoxide synthetase (iNOS), cyclooxygenase-2 (COX-2), and a placenta growth factor. A cell sheet containing such a factor facilitates angiogenesis at the engrafted site.

### EXAMPLES

The present technology will now be described in detail by way of Examples. The following Examples should not be construed to limit the scope of the present technology. The experimental protocol in Examples has been approved by the ethics committee on animal study in Tokyo Women's Medical University. The experiments were performed in accordance with Guide for the Care and Use of Laboratory Animals (revised edition, 1996) published by National Institutes of Health (NIH) in the USA.

### Animals, Reagents, and Kits Used

GFP transgenic rat (Sankyo Labo Service Corporation, INC)

### Sprague-Dawley rat (Sankyo Labo Service Corporation, INC)

### Penicillin/streptomycin (FUJIFILM Wako Pure Chemical Corporation, #168-23191)

Fetal bovine serum (FBS; Life Technologies, #10270-106)

### Trypsin-EDTA (X1) (FUJIFILM Wako Pure Chemical Corporation, #208-17251)

L-ascorbic acid phosphate ester magnesium salt n-hydrate (FUJIFILM Wako Pure Chemical Corporation, #013-19641)
Povidone-iodine (Isocline (registered trademark), Meiji Seika Pharma Co., Ltd. #50400)

### Collagenase (SERVA, #17465 NB 4G Proved Grade)

Distilled water (Otsuka Pharmaceutical Co., Ltd.)
RNeasy (registered trademark) Fibrous Tissue Mini Kit (QIAGEN, #74704)

### Mitomycin C (Wako Pure Chemical Industries, Ltd., #134-07911)

Hepatocyte Growth Factor (Heapapoietin A, Scatter Factor) (HGF) ELISA Kit (antibodies,online.Com, #ABIN367412)
FGF basic Pig ELISA Kit (Abcam PLC, #ab156467)
D-MEM (high glucose) (FUJIFILM Wako Pure Chemical Corporation, #043-30085)

### Laboratory Ware

Flask (75 cm²) (BD Falcon, #353810)
Temperature-responsive culture plate (35 mm) (UpCell (registered trademark)) (CellSeed Inc., #CS3007)

### 1. Isolation and Cultivation of Adipose-Derived Stem Cells and Preparation of Cell Sheet

### (1) Isolation of Adipose-Derived Stem Cells

Adipose-derived stem cells were isolated in accordance with Watanabe N. et al. “Genetically Modified Adipose Tissue-Derived Stem/Stromal Cells, Using Simian Immunodeficiency Virus-Based Lentiviral Vectors, in the Treatment of Hemophilia” B. Hum Gene Ther. 2013 March; 24 (3): 283-294.

Subcutaneous fat tissues (20 g) at groins were collected with local anesthesia from GFP transgenic rats so as not to contain blood cell components as much as possible. The collected fat tissues were disinfected with povidone-iodine and then were rinsed two times with an antibiotic-containing culture medium (D-MEM containing 1% penicillin-streptomycin). After rinsing, the tissues were cut into small pieces on a dish with scissors. Each of 4 g aliquots was placed into a 50 mL tube, 35 mL of antibiotic-containing culture medium was added, and then 1 mL of collagenase (concentration: 0.27 pzu/mL) was added to each sample. After the mixture was agitated for one hour at 37° C. and 130 rpm, it was subjected to centrifugal separation for five minutes at 4° C. and 2000 rpm. The tube was manually shaken for 30 seconds. The mixture was subjected to centrifugal separation again for five minutes at 4° C. and 300 G. Large tissue pieces floating on the surface of the tube are removed, and the solution was filtered through a cell strainer (100 μm) (#352360 available from Japan Becton, Dickinson and Company) and then another cell strainer (40 μm) (#352340 available from Japan Becton, Dickinson and Company). The solution was subjected to centrifugal separation for five minutes at 4° C. and 1500 rpm. After the supernatant was removed, the pellet was suspended in a culture medium containing 10% FBS. The suspension was seeded onto five flasks (75 cm²) and cultured at 37° C. in an incubator.

### (2) Cultivation of Adipose-Derived Stem Cells and Preparation of Cell Sheet

The culture medium was replaced on the third day after the cell seeding. After the cells were separated in 0.25% trypsin on the fifth day, and they were subcultured on ten 75 cm² flasks. After two or three days, the cells were re-subcultured, and then they were separated in 0.25% trypsin on the second or third day. After cell counting, 2.3×10⁶ cells were suspended in a 2 mL culture medium. The suspension was seeded on 35 mm UpCell (registered trademark) and subjected to incubation at 37° C. for two days. After two days, the culture medium was replaced with new one containing 16.4 μg/mL ascorbic acid. After two more days, the culture medium was again replaced with new one containing ascorbic acid, and cells were incubated for 20 to 30 minutes in an incubator at 20° C. to recover cells in a sheet form immediately before the implantation of the cell sheet.

### (3) Preparation of Sheet for Covering Wound

Peritoneal membranes were collected from SD rats and were laminated on the cell sheet produced in the preceding process to prepare sheets for covering a wound.

### 2. Implantation of Sheet for Covering Wound and Cell Sheet

The fascia in the central back of an SD rat was removed to expose muscles. The sheet for covering a wound prepared in the preceding process was engrafted into the back muscle by four-times sutures. For purposes of comparison, only a cell sheet or only a peritoneal membrane was engrafted into a back muscle by pasting.

### 3. Visual Evaluation

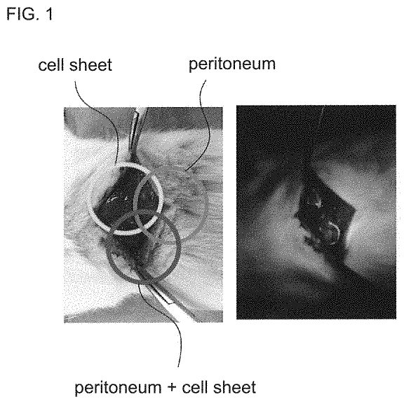

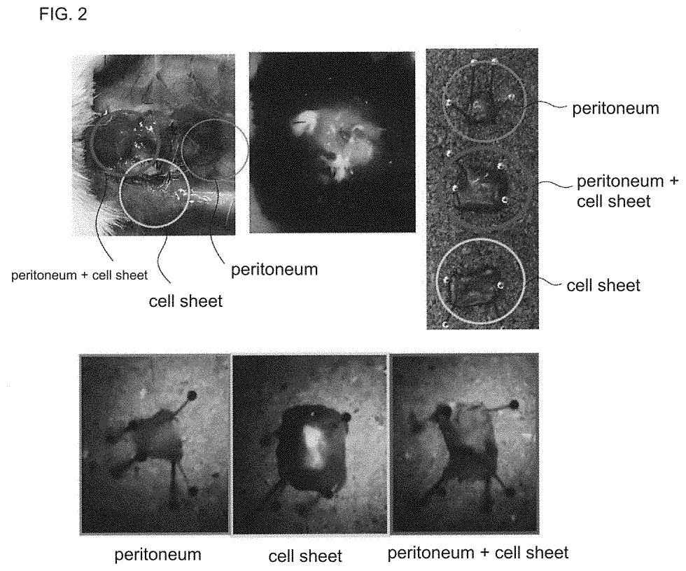

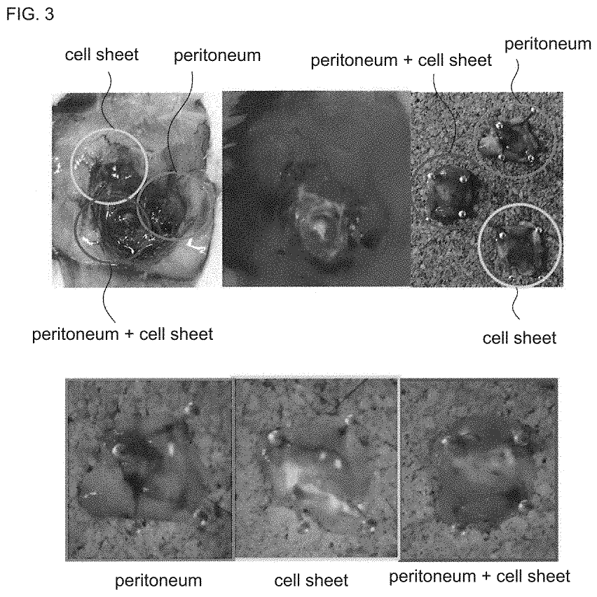

The state of each back muscle after the implantation was observed immediately after the surgery, and on the third day and the seventh day after the surgery. FIG. 1 includes photographs of the back muscles immediately after the implantation of the sheet for covering a wound, only the cell sheet, and only the peritoneal membrane. FIG. 2 includes photographs of the back muscles on the third day after the implantation of the sheet for covering a wound, only the cell sheet, and only the peritoneal membrane. FIG. 3 includes photographs of the back muscles on the seventh day after the implantation of the sheet for covering a wound, only the cell sheet, and only the peritoneal membrane.

FIG. 2 demonstrates that the cell sheets remain at the engrafted site of the sheet for covering a wound and the engrafted site of only the cell sheet on the third day after the surgery, and that the cell sheet slightly flows in the engrafted site of only the cell sheet.

FIG. 3 demonstrates that the cell sheet for covering a wound including a peritoneal membrane and a cell sheet maintains its original shape at the engrafted site whereas the cell sheet almost flows out from the site into which only the cell sheet is engrafted, on the seventh day after the surgery. Furthermore, the cell sheet for covering a wound including a peritoneal membrane and a cell sheet is stably engrafted compared with the site into which only the cell sheet is engrafted.

### 4. Histological Evaluation

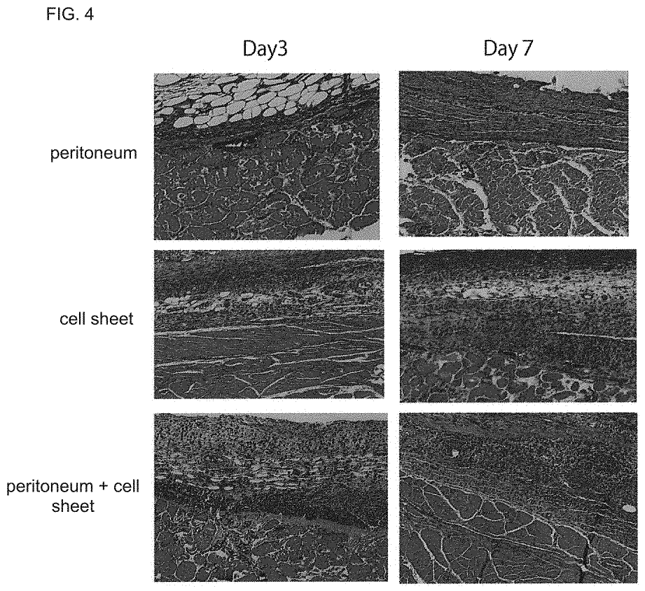

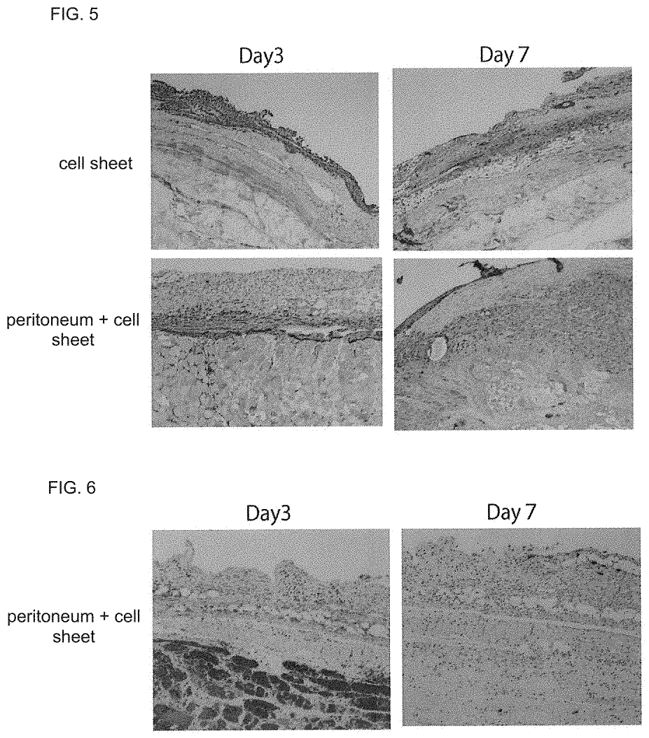

Samples excised on the third day and the seventh day after the surgery were each embedded into a compound, which was then frozen with fluid nitrogen to prepare a tissue section. Each section was stained with hematoxylin-eosin, green fluorescent protein (GFP), or calretinin. FIG. 4 includes hematoxylin-eosin-stained microscopic photographs. FIG. 5 includes GFP-stained microscopic photographs. FIG. 6 includes calretinin-stained microscopic photographs.

The cell sheet is positive for both the GFP stain and the calretinin stain. As shown in FIGS. 5 and 6, the cell sheet is stably engrafted at the site into which the sheet for covering a wound including the peritoneal membrane and the cell sheet is engrafted even on the seventh day after the surgery.

1. A sheet to be engrafted into a wound region to cover a wound, the sheet comprising:
a serosal membrane having an implant face; and
a cell sheet laminated onto the implant face of the serosal membrane.

2. The sheet for covering the wound according to claim 1, wherein the serosal membrane is a peritoneal membrane.

3. The sheet for covering the wound according to claim 1, wherein the wound region comprises a sutured or inosculated wound region.

4. The sheet for covering the wound according to claim 1, wherein the wound region comprises a wound region of a hollow organ.

5. The sheet for covering the wound according to claim 1, wherein the sheet is engrafted into the outer wall of a hollow organ.

6. A method of covering a wound, comprising:
engrafting a sheet for wound therapy into a wound region, wherein the sheet comprises a serosal membrane having an implant face and a cell sheet laminated onto the implant face of the serosal membrane.

7. The method of covering a wound according to claim 6, wherein
the sheet for wound therapy is engrafted by saturation into the wound region.

8. The method of covering a wound according to claim 6, wherein
the serosal membrane is a peritoneal membrane.

9. The method of covering a wound region according to claim 6, wherein
the wound region comprises a sutured or inosculated wound region.

10. The method of covering a wound according to claim 1, wherein
the wound region is a wound region of a hollow organ.

11. The method of covering a wound according to claim 6, wherein
the sheet for wound therapy is engrafted into the outer wall of a hollow organ.
